(19) United States
(12) Patent Application Publication
(10) Pub. No.: US 2007/0259327 A1
(43) Pub. Date: Nov. 8, 2007

(54) Cryopreservation medium and cryopreservation method for tissues and cells

(75) Inventors: Yasuhiro Iwanaga, Sakyo-ku (JP); Kazuaki Matsumura, Kamigyo-ku (JP); Jong-Yoon Kim, Kamigyo-ku (JP); Hajime Sugai, Kamigyo-ku (JP); Suong-Hyu Hyon, Sakyo-ku (JP)

Correspondence Address: JORDAN AND HAMBURG LLP, 122 EAST 42ND STREET, SUITE 4000, NEW YORK, NY, 10168, US

(73) Assignee: BioVerde. Inc., Higashigawa (JP)

(21) Appl. No.: 11/418,421

(22) Filed: May 3, 2006

(51) Int. Cl. C12N 5/08; A01N 1/02

(52) U.S. Cl. 435/1.1; 435/2; 435/366

(57) ABSTRACT

A cryopreservation medium and a cryopreservation method which make it possible to cryopreserve tissues and cells with high viability are provided. A cryopreservation medium contains polyphenol at a concentration of from 30 to 120 ppm. In an embodiment, a cryopreservation method for tissues and cells comprises: incubating tissues or cells for 2 hours in culture medium supplemented with polyphenol at concentrations of from 30 to 120 ppm; suspending tissues or cells in a cryopreservation medium; freezing the suspension of the tissues or cells in the cryopreservation medium; and storing the frozen suspension of tissues or cells in the cryopreservation medium.

### FIELD OF THE INVENTION

The present invention relates to a cryopreservation medium and a cryopreservation method for tissues and cells, which have the potential to promising applications in the fields of cell culture, cell and tissue transplantation and research for embryonic stem (ES) cells

### BACKGROUND OF THE INVENTION

Dimethyl sulfoxide (DMSO) is the most frequently used cryoprotectant in islet cell cryopreservation. The cryopreservation method used most frequently is slow cooling to −40° C. and rapid thawing from −196° C.¹.

However, the cells established by the above method have a problem of poor cryopreservation efficiency under the conventional cryopreservation conditions mainly used for islets cells.

Polyphenol has several bioactivity like antioxidant, radical scavenger and anti-apoptosis besides antimicrobial, deodorant, anti-cancer and anti-viral². These bioactivity is thought to be effective to prevent from freeze and thaw injury.

The object of the present invention is to provide a cryopreservation medium and a cryopreservation method which make it possible to cryopreserve tissues and cells with high viability.

### BRIEF SUMMARY OF THE INVENTION

The present inventors have carried out studies to solve the above-mentioned problems and have completed the present invention on the basis of the finding that the use of a cryopreservation medium containing polyphenol as a cryoprotectant at a concentration of from 10 to 300 ppm, allows cryopreservation of tissues and cells with high viability.

### BRIEF DESCRIPTION OF THE SEVERAL VIEWS OF THE DRAWING

FIG. 1 shows recovery rates of islets following cryopreservation and after 48 hours of postcryopreservation tissue culture in 4 groups in Example.

FIG. 2 is islet viability of post-cryopreservation islets in 4 groups in Example.

FIG. 3 is functionality of post-cryopreservation islets in 4 groups in Example.

### DETAILED DESCRIPTION OF THE INVENTION

Materials and Methods

Islets were isolated from over two years old pigs (n=3). After overnight culture, a known number of islets were incubated for 2 hours in culture medium supplemented with polyphenol (epigallocatechin-gallate (EGCg) with more than 98% purity) at concentrations of 0 (control), 30, 60, 120 ug/ml. Islets were suspended in the same volume of cryopreservation solution containing 12% hydroxyethyl starch (HES) and 10% dimethyl sulfoxide (DMSO) with 8% fetal blood serum (FBS), and then cryopreserved using a program freezing system³. After 4 weeks storage in liquid nitrogen, islets were rapidly thawed in a hot water bath. We assessed the results on recovery rate (islet yield after thawing/islet yield before freezing) following cryopreservation and after 48 hours of postcryopreservation tissue culture, viability and functionality.

### EXAMPLE

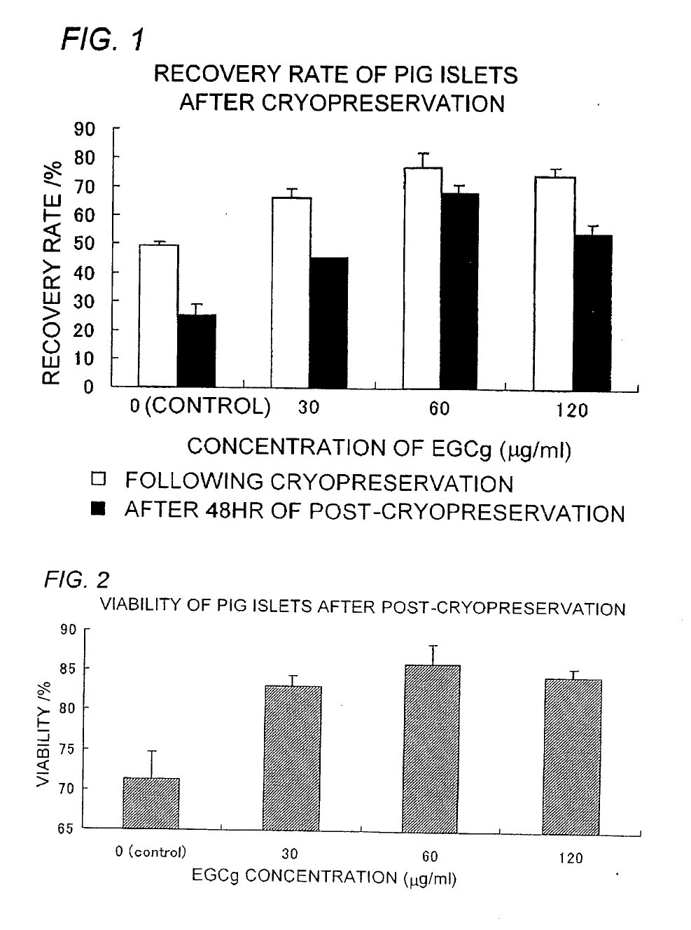

As shown in FIG. 1, islet recovery rates following cryopreservation were 49.3±1.6%, 66.5±3.3%, 76.9±5.2%, 74.5±2.9% in groups 0, 30, 60, 120 ug/ml of polyphenol, respectively. Islet recovery rates after 48 hours of postcryopreservation tissue culture were 25.2±3.6%, 45.3±0.3%, 68.0±3.0%, 54.6±3.3%, respectively. Islet recovery following cryopreservation in the polyphenol group were significantly higher than the control group (p<0.01).

As shown in FIG. 2, viability assessed using AO/PI were 71.3±3.4, 83.1±1.2, 85.9±2.5, 84.4±1.0 respectively. Viability following cryopreservation in the polyphenol group were significantly higher than the control group (p<0.01).

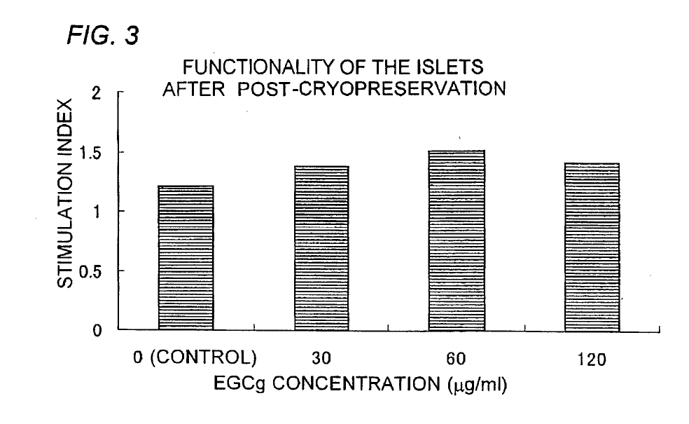

As shown in FIG. 3, Functionality indicated by stimulation index was 1.2±0.1, 1.4±0.2, 1.4±0.3, 0.9±0.3, respectively. The stimulation index represents a ratio of insulin secretion between high-concentration glucose culture medium and low-concentration glucose culture medium.

### LIST OF REFERENCES CITED

1. Corominola H, Mendola J, et al. Cryopreservation of pancreatic islets prior to transplantation: A comparison between UW solution and RPMI culture medium. CRYOBIOLOGY 1998; 37: 110-118.
2. Hyon, S.-H. et al. (2005) Protection of human fibroblasts from reactive oxygen species by green tea polyphenolic compounds. Key Engineering Materials 288-9:665-668.
3. Maruyama M, Kenmochi T, et al: Simplified method for cryopreservation of islets using hydroxyethyl starch and dimethyl sulfoxide as cryoprotectants. Transplantation Proceedings 2004; 36: 1133-1134.

1-2. (canceled)

3. A cryopreservation medium for tissues and cells for transplantation effective in preventing freeze and thaw injury, comprising polyphenol as a cryoprotectant at a concentration of from 30 to 120 ppm (parts per million) and further comprising dimethyl sulfoxide and/or glycerin as a cryoprotectant.

4. A cryopreservation medium for tissues and cells for transplantation effective in preventing freeze and thaw injury, comprising polyphenol as a cryoprotectant at a concentration of from 30 to 120 μm, and further comprising dimethyl sulfoxide as a cryoprotectant at a concentration from 5% (W/V) to 20% (W/V).

5. A cryopreservation medium for tissues and cells for transplantation effective in preventing freeze and thaw injury, comprising polyphenol as a cryoprotectant at a concentration of from 30 to 120 ppm, and further comprising hydroxyethyl starch (HES) as a cryoprotectant at a concentration from 5% (W/V) to 20% (W/V).

6. (canceled)

7. A cryopreservation medium for tissues and cells for transplantation effective in preventing freeze and thaw injury, comprising polyphenol as a cryoprotectant at a concentration of from 30 to 120 ppm, and further comprising, in addition to the cryoprotectant, serum and/or a serum replacement, a basal medium for animal tissue culture containing at least from 0.3% (W/V) to 5% (W/V) of glucose, at a concentration of from 10% (W/V) to 75% (W/V).

8-9. (canceled)

10. A cryopreservation method for islets isolated from humans and animals comprising: incubating said islets in culture medium supplemented with polyphenol at concentrations of from 10 to 300 ppm; suspending said islets in a cryopreservation medium; freezing the suspension of said islets in the cryopreservation medium by cooling them; and storing the frozen suspension of said islets in the cryopreservation medium.

11.-13. (canceled)
